(19) United States
(12) Patent Application Publication
(10) Pub. No.: US 2005/0101843 A1
(43) Pub. Date: May 12, 2005

(54) Wireless disposable physiological sensor

(75) Inventors: David E. Quinn, Weedsport, NY (US); Ray D. Stone, San Diego, CA (US); John Lane, Weedsport, NY (US); Frederick F. Schweitzer JR., Cicero, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc.

(21) Appl. No.: 10/702,631

(22) Filed: Nov. 6, 2003

(51) Int. Cl. A61B 5/00

(52) U.S. Cl. 600/300; 340/539.12; 374/142; 340/573.1

(57) ABSTRACT

A patient physiological parameter monitoring apparatus for a subject includes a sensor assembly having at least one responsive element that produces a first signal upon detection of a change in temperature. The assembly also includes a converter for converting the first signal into an electrical signal, and a transmitter for wirelessly transmitting converted electrical signals upon demand based upon receipt of a transmitted signal from an interrogation device. Preferably, at least a portion of the sensor assembly is disposable to permit single use or single subject use and can be further used to track location and information of medical equipment in addition to subjects.

### FIELD OF THE INVENTION

This invention relates generally to the field of diagnostic medicine, and more specifically to medical diagnostic apparatus including a wireless sensor assembly that passively measures the body temperature or other physiological parameter of a subject or relates to a situated diagnostic device for locating either or relation to a device specific characteristic wherein at least a portion of the sensor assembly is disposable.

### BACKGROUND OF THE INVENTION

Thermometers are commonly known in the medical field for measuring the core body temperature of a patient. In the majority of these devices, a probe that contains or retains at least one temperature measuring or sensing element, such as a thermocouple or thermistor, is placed at a body site such as the sublingual pocket, or alternately the axillary area, rectal cavity or within the ear canal. The temperature sensing element then either predicts temperature or is caused to remain at the body site until the sensing element reaches the environment temperature after which the probe is either removed for reading by the user or the measured reading is displayed.

Alternatively, the thermometer can include a resistive or other form of heater used to preheat the temperature sensing element to that which is somewhat closer to the temperature of the body site in order to effectively hasten reading/measurement time.

In addition to the above wired thermometry devices, there are such as those described in U.S. Pat. Nos. 5,252,962 and 6,054,935 to Urbas et al. that effectively remove the “tether” between the control unit and the probe assembly. To date, such devices are found only for use in certain veterinary applications and only in relation to implantable devices.

### SUMMARY OF THE INVENTION

It is therefore a primary object of the present invention to provide a versatile, disposable, low-cost patient temperature or other physiological parameter measuring device.

It is another primary object of the present invention to provide a wireless physiological parameter measuring device, such as a thermometer, that continually measures patient body temperature and which can be accessed on demand.

Therefore and according to a preferred aspect of the present invention, there is disclosed an apparatus for measuring at least one physiological parameter of a patient, said apparatus comprising:
a sensor assembly including at least one physiological parameter responsive element, said responsive element producing a first signal upon detection of a change in physiological parameter, a converter for converting the first signal into an electrical signal, and a transmitter for wirelessly transmitting the converted electrical signal upon demand; and
an interrogation device having a transmitter that wirelessly transmits a signal to said sensor assembly, wherein said sensor assembly does not transmit readings of said at least one physiological parameter responsive element until the interrogation device transmits the signal, and in which said sensor assembly is disposed on a patient to enable physiological parameters readings to be taken without significant delay, and in which at least a portion of said sensor assembly is disposable.

Preferably, because at least a portion of the sensor assembly is disposable, it can selectively be dedicated for single use or single patient use and/or can be used a discrete number of times.

The sensor assembly can include a programmable ASIC that permits information, such as patient or device-related data including demographics including date of birth, insurance carrier information, family medical history, etc., to be stored for subsequent access by the interrogator device to permit this information to track along with the patient, for example, in a hospital or physician's office encounter. Preferably, the ASIC is attached to a back surface of a disposable sensor assembly wherein at least a portion of the sensor assembly is discarded after patient use, while the ASIC snaps off or is otherwise releasably removed and can be reused. For example, a temperature sensor assembly using the inventive concepts described herein can be made into a flexible assemblage, utilizing technologies, such as silk screening of at least portions thereof, such as the battery, antenna and thermistor, permitting single use or single or multiple patient use.

The present device is also capable of measuring different physiological parameters including but not limited to blood gas, SPO₂, blood pressure and heart rate in addition to or in lieu of body temperature. In order to accomplish this objective, various bio-sensors can be attached to the present assembly to permit multiple uses and versatility thereof. Due to the proximity of the sensor assembly to the subject, the device would operate effectively as a monitor and not, for example in the case of body temperature, as a “predict” temperature apparatus.

The readings obtained by the device can be archived or stored and can be data logged, permitting temperature/pulse and other useful parameter trend data/analysis.

According to another variant of the invention, a temperature sensor assembly employens the inventive concepts described herein can be disposed within a wraparound disposable apparatus, such as an inflatable blood pressure cuff, that can be wrapped around a limb (e.g., the arm or leg) of a subject.

Depending upon its construction, the parameter sensor assembly can include multiple parameter responsive elements or can be applied conveniently on different parts of the subject. For example, a pair of temperature sensing assemblies can be attached to a subject to determine thermal variations; for example, the breaking of a limb, a blood clot, or other perceivable problem in an extremely simple and convenient manner.

In addition, the present device can be further utilized for other applications. For example, the device could be implanted near cancerous tumors and include a sensor enabling same to be able to measure radiation dosages at a specific site. This detection could be used effectively to determine correct dosages of radiation therapy. By its convenience in size, the parameter measuring assembly is not limited to on-the-body measurements, meaning the device can be conveniently attached or implanted, used, as needed, for monitoring purposes and then removed at the end of treatment.

The herein described measuring apparatus can further be used to monitor stress in vascular and arterial walls on a real-time basis by implantation near glands and be able to measure secretions that are, for example, doped with a tracer element at a specific site. These measurements could be taken before they interact with other fluids or as real-time collection of data, such as, for example, drug delivery and other treatments, or to track subject location.

According to another preferred aspect of the invention, there is provided a wireless thermometer apparatus for measuring the body temperature of a subject, said thermometer comprising:
a sensor assembly including at least one temperature responsive element that produces a first signal upon detection of a change in body temperature, a converter for converting the first signal into an electrical signal, and a transmitter for wirelessly transmitting electrical signals upon demand; and
an interrogation device having a transmitter that wirelessly transmits a signal wherein said sensor assembly does not transmit readings of said at least one temperature responsive element until the interrogation device transmits said signal, and in which said sensor assembly is disposed on a subject to enable temperature readings to be taken without significant delay, wherein at least a portion of said sensor assembly is disposable.

According to yet another preferred aspect of the present invention, there is disclosed a method for measuring at least one physiological parameter of a subject, said method comprising the steps of:
attaching a disposable sensor assembly to the body of a subject, said sensor assembly including at least one physiological parameter sensor that is responsive to a trigger signal;
selectively transmitting a trigger signal in the vicinity of said sensor assembly;
said sensor assembly transmits a reading from said sensor only in response to reception of said trigger signal and in which at least a portion of said sensor assembly is disposable.

According to still another preferred aspect of the present invention, there is disclosed a method for identifying the location of medical equipment in a subject's room, said method including the steps of:
attaching a sensor assembly to at least one piece of medical equipment, said sensor assembly including a programmable ASIC that includes information concerning said equipment stored therein, a wireless transmitter, a wireless receiver and an antenna for permitting bi-directional wireless communication;
selectively transmitting a trigger signal from an interrogation device in said subject's room; and
transmitting product information from said sensor assembly to said interrogation device only in response to said trigger signal and in which at least a portion of said sensor assembly is disposable.

As noted, at least a portion of the sensor assembly is disposable and is preferably made from a flexible strip that can be easily attached through adhesive or other means to the equipment for tracking or inventory purposes. This function is useful for billing purposes as well in settling disputes as to whether a price of equipment or procedure was performed on a subject. The function is also useful for traceability and for marrying of physiological data to a piece(s) of equipment, such as but not limited to calibration data.

The disposability aspect of the present invention provides ease of use for patient application and less risk of cross contamination between subjects or patients.

One advantage realized by the present invention is that temperature or other physiological parameters can be obtained on demand almost instantaneously. Therefore, realizable time savings are achieved by the present measuring apparatus.

Another advantage provided is that the present assembly is entirely wireless, thereby avoiding cumbersome cables, wires or connectors and providing convenience and versatility for the subject, patient and caregiver.

Another advantage is that the sensor assembly has relatively low mass as well as high flexibility. The sensor assembly can also be attached to any piece of equipment, such as a vital signs monitor or other device found in a patient's room, permitting the sensor assembly to be used in order to track the location of apparatus as part of inventory control, or in detecting whether an instrument is present in the patient room using the interrogation device.

These and other objects, features and advantages will become apparent from the following Detailed Description which should be read in conjunction with the accompanying drawings.

### BRIEF DESCRIPTION OF THE DRAWINGS

FIG. 1 is a perspective view of a physiological parameter measuring apparatus in accordance with a preferred embodiment of the present invention;

FIG. 2 is a bottom view of a disposable sensor assembly in accordance with a preferred embodiment for use with the physiological parameter measuring apparatus shown according to FIG. 1;

FIG. 3 is a perspective view of the disposable sensor assembly of FIG. 2;

FIG. 4 is a bottom view of a disposable sensor assembly in accordance with an other preferred embodiment of the present invention;

FIG. 5 is a perspective view of the disposable sensor assembly of FIG. 5;

FIG. 6 depicts a generalized functional schematic diagram of the temperature measuring apparatus of FIGS. 1-6;

FIG. 7 depicts a top perspective view of an interrogator device in accordance with a preferred embodiment for use with the physiological parameter measuring apparatus of FIG. 1;

FIG. 8 illustrates an alternate embodiment of the physiological parameter measuring apparatus as used in conjunction with a blood pressure sleeve;

FIG. 9 depicts an alternate application of the physiological parameter measuring apparatus for use with a patient;

FIG. 10 is an alternate embodiment of the above measuring apparatus as used for purposes of an equipment inventory or tracking control function; and

FIG. 11 depicts yet another alternate application of the above measuring apparatus as used with an endoscopic apparatus.

### DETAILED DESCRIPTION

The following description relates to certain preferred embodiments and applications of a patient physiological parameter measuring apparatus made in accordance with the present invention. It will be readily apparent to one of sufficient skill in the field, however, that there are modifications and variations that can be implemented within the intended scope of the invention. In addition, and throughout the course of this description, certain terms are used to provide in order to assist the reader and to provide a frame of reference with regard to the accompanying drawings. These terms, however, should not be interpreted as overly limiting to the intended scope of the inventive concept, except where specifically indicated.

Referring to FIG. 1, there is shown a wireless patient monitoring or measuring apparatus 10 made in accordance with a preferred embodiment of the present invention. This wireless monitoring apparatus 10 includes a patient sensor assembly 20 and an interrogation device 30. The patient sensor assembly 20 is preferably at least partially disposable and is removably attachable to the skin of a subject and can be conveniently applied anywhere thereupon, the sensor assembly including a gel or adhesive pad for permitting direct attachment to the skin. In this instance, the sensor assembly 20 is shown as being attached to the neck area of the patient 34. As detailed in a later portion, however, it will be shown that the sensor assembly 20 is not limited to merely subject attachment and that there are numerous examples of other varied uses and applications therefor.

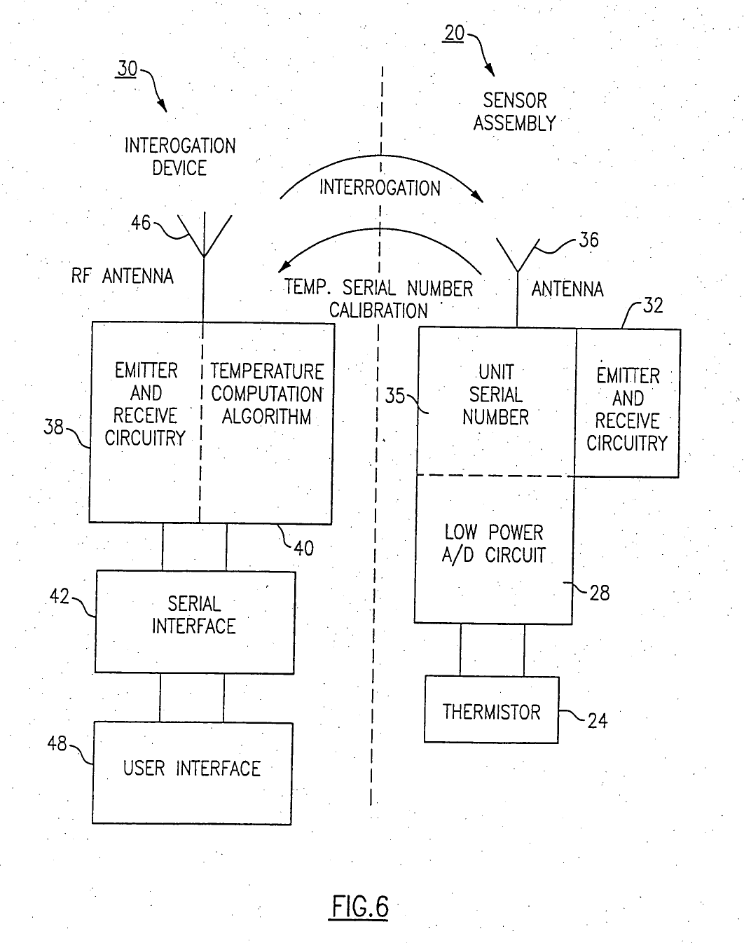

As shown schematically in FIG. 6, the sensor assembly 20 according to this embodiment includes at least one element or sensor that is responsive to a physiological parameter. In this embodiment, at least one temperature responsive element 24, such as a thermistor, thermocouple, or other miniature temperature responsive sensor is provided, the sensor being electronically coupled to low power circuitry that includes analog to digital conversion for converting an electrical signal generated by the temperature responsive element into a digital signal that can be transmitted to the interrogation device 30. Power for the sensor assembly 20 is created through a passive connection magnetically due to a generated trigger signal from the interrogation device 30, the sensor assembly including a power generation/power control block. Alternately, the block can be configured to permit active powering of the sensor assembly 20 upon receipt of the trigger signal or that the sensor assembly remains active irrespective of whether a trigger signal is transmitted by the interrogation device.

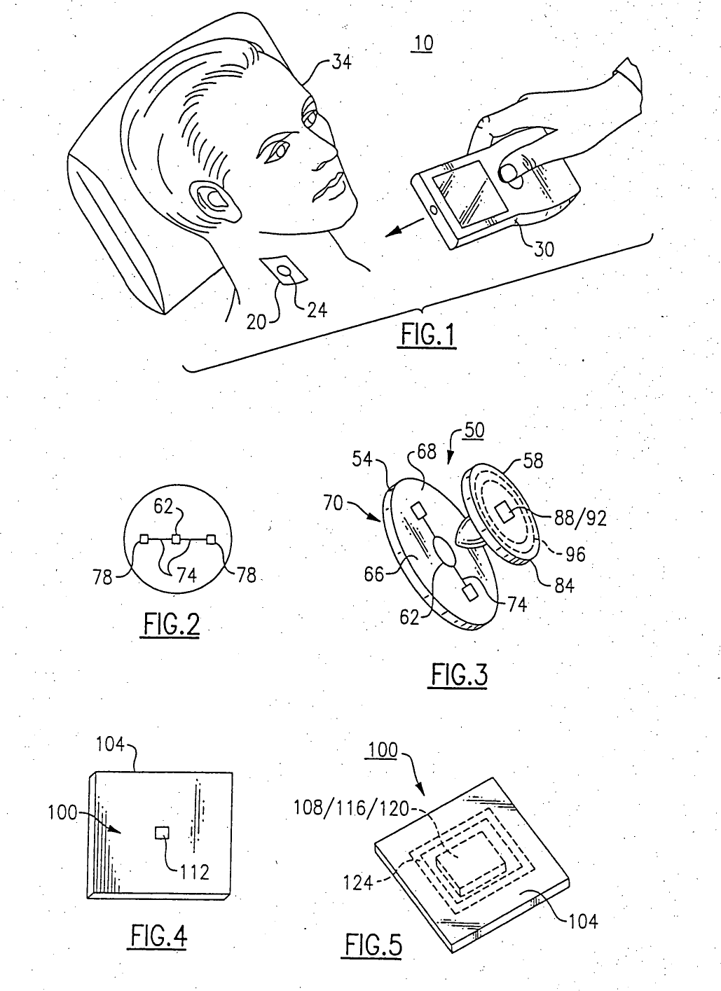

The sensor assembly 20 further includes circuitry for routing the digital signal by means of wireless emitter and receive circuitry 32 that permits the processed signal to be transmitted wirelessly by means of an antenna 36 to the interrogation device 30. Each of the above components are preferably included in the sensor assembly in a patch-like configuration. Examples of specific sensor assemblies are further described herein with reference to FIGS. 2-5.

According to a first embodiment, shown in FIGS. 2 and 3, a disposable two piece sensor assembly 50 includes a first disposable supporting portion 54 and a second reusable portion 58. By “disposable”, it is meant that the portion can be discarded after a single use or after a single patient use and replaced. The first disposable portion 54 of this assembly includes a temperature responsive element 62, such as a thermistor, that is bonded to a flexible strip 66 having an adhesive backing 70. Embedded within the flexible strip 66 are leads 74 extending from the temperature responsive element 62 to a pair of connection coupling pads 78. The reusable section 58 of this assembly 50 includes a body portion 84 that is manufactured from a lightweight plastic material and preferably includes an embedded programmable ASIC 88, as well as a wireless transmitter/receiver 92 and an antenna 96, wherein the reusable portion 58 is preferably releasably attached to the top surface 68 of the flexible strip 66. Preferably, the disposable supporting portion 54 is manufactured using silk screen or other technology.

According to an alternate embodiment, as shown in FIGS. 4 and 5, a second type of sensor assembly 100 in accordance with the invention can be manufactured as a single piece, for preferably either disposable or single subject use. According to this embodiment, the sensor assembly 100 is defined by a flexible substrate 104 that includes a programmable ASIC 108 that is embedded, along with a thermistor 112, acting as the temperature responsive element, as well as a wireless transmitter 116, a wireless receiver 120 as well as an antenna 124, each operatively interconnected. As noted above, other forms of temperature responsive elements can be substituted. In each of these sensor assemblies, unit device or serial information, shown diagrammatically as block 35 in FIG. 6, can be stored into the programmable memory of the ASIC 108 such that both parameter data as well as unit/tag information can be transmitted to the interrogation device 30 following reception of the trigger signal by the sensor assembly 100.

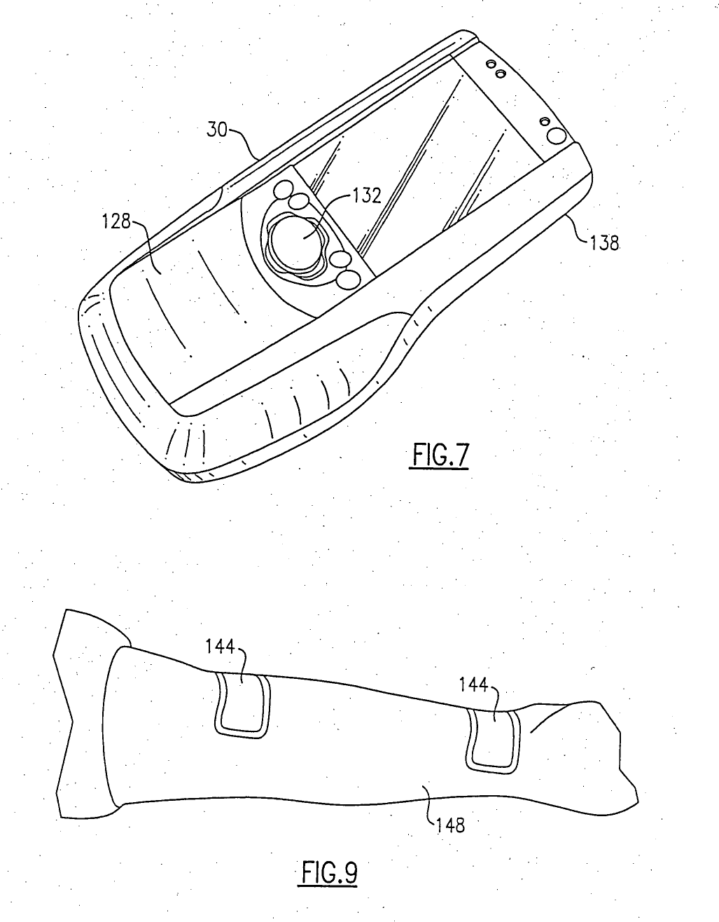

Referring to FIGS. 1, 6 and 7, the interrogation device 30 is preferably a hand-held device, such as a PALM-type device or personal data assistant (PDA), that also includes onboard transmit and receive circuitry 38 in the form of a wireless transceiver, in order to enable wireless communication with the sensor assembly 20, as well as a corresponding antenna 46. According to the present invention, the form of wireless communication between the sensor assembly 20 and the interrogation device 30 is via RF (radio frequency) generation, though other methods of wireless communication, including but not limited to optical, ultrasonic, and infrared could similarly be utilized. The interrogation device 30 further includes a user interface 48 that includes a display, such as an LCD 128, as well as input controls 132 on a facing surface of a device housing 138, for operating same. For example, threshold alarm limits can be set or programmed by the device whereby readings that exceed a predetermined level will cause an alarm to be triggered.

Still referring to FIG. 6, the interrogation device 30 further includes a miniature processor that includes at least one stored temperature computation algorithm(s) as well as calibration data that is used in conjunction with the readings obtained from the sensor assembly 30. The processor is interconnected to a serial interface 42 that is connected to the user interface 48.

Essential to the operation of the above described assembly, is that the sensor assembly 20 operates passively until a trigger signal is selectively transmitted from the interrogation device 30 and received by the sensor assembly. Upon receipt of this signal, energy is collected and conditioned to temporarily power the sensor assembly 20. Therefore, reading from the temperature responsive element 24, which is proximity with the area of interest and is active throughout, is obtained. The reading is converted into an electrical signal which is then wirelessly transmitted to the interrogator device 30.

As shown in FIG. 9, it should be readily apparent that more than one sensor assembly can be placed or positioned for use on a subject. There are instances when a plurality of sensor assemblies 144 can be attached to the arm 148 or other area of a subject, for example, to determine if there is a broken bone, a blood clot, or other injury when thermal variations can be determined to locate same.

Though the preceding has been shown with a single form of miniature parameter (e.g., a temperature) sensor, it should be readily apparent that at least one other form of physiological parameter sensor, such as, but not limited to heart rate, SPO2, and respiration can be attached to the sensor assembly.

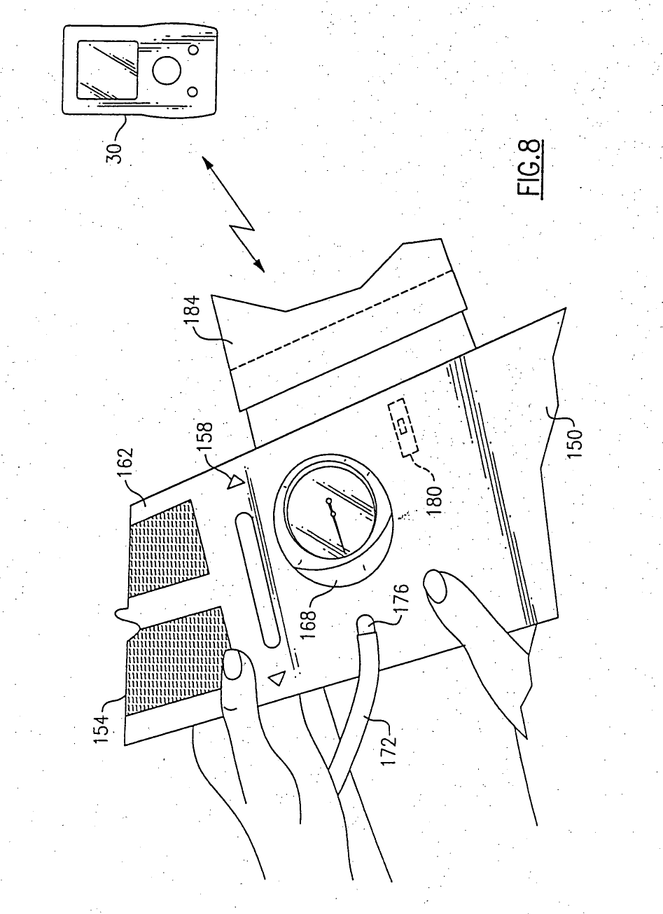

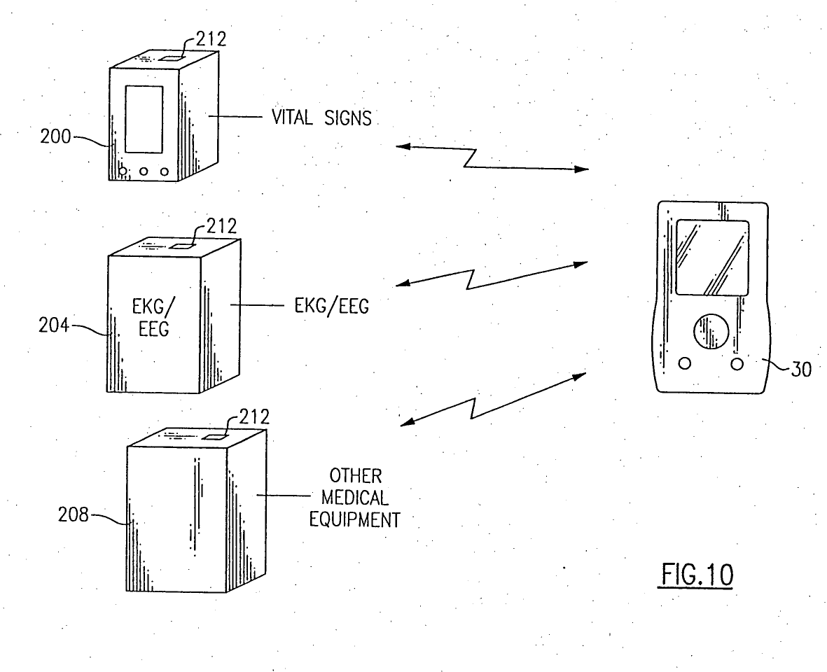

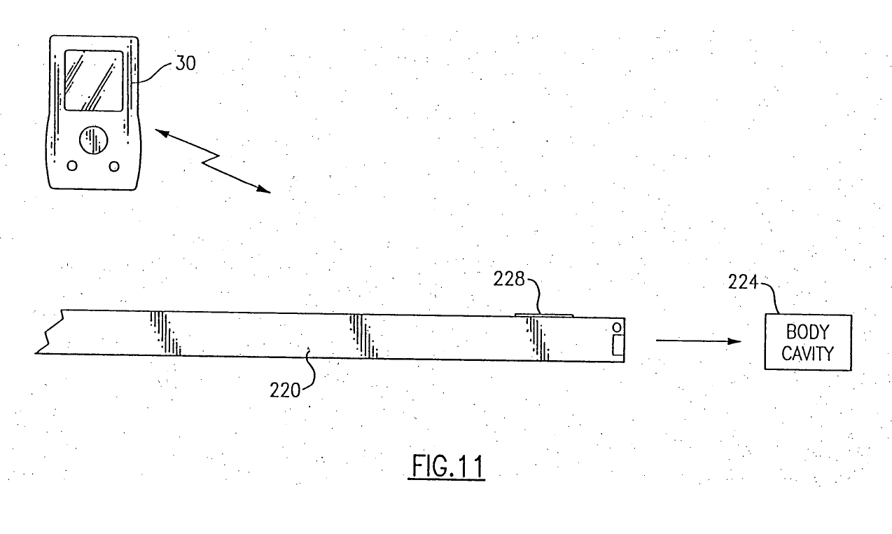

Several applications of the above disposable sensor assembly are possible. For example and referring to FIG. 8, one potentially useful application for the herein described sensor assembly is in connection with an inflatable blood pressure cuff or sleeve 150. The sleeve 150 is wrappable about a limb of a subject and includes hook and loop fasteners 154 that permit an adjustable securement of the sleeve. A top facing side 162 of the sleeve 150 shown includes artery markers 158 that are used to align the sleeve with the brachial artery of the arm 184 of the subject, as well as a socket (not shown) permitting direct connection of a gage housing 168. The sleeve 150 further includes a hose 172 attached to a pneumatic bulb (not shown) that is fluidly connected to the interior of the sleeve 150 through a coupling 176. A sensor assembly 180, such as those previously described above and shown in phantom, is attached to the bottom facing side of the sleeve (that is, the side facing the subject) wherein temperature readings can be selectively gathered in conjunction with blood pressure readings using the sensor assembly in the manner previously described using interrogation device 30. Alternately and as further detailed below, the sensor assembly 180 can be attached to any portion of the sleeve 150 and used to detect the presence of the sleeve via transmission of an identification signal in response to the trigger signal transmitted by the interrogation or other suitable device. This function is described in greater detail below with reference to FIG. 10.

This inventory and/or tracking method is more fully depicted according to FIG. 10 in which several instruments or apparatus in a patient room can be located and identified by an interrogation device 30 upon a caregiver entering the room. In this embodiment, several pieces of equipment, such as a vital signs monitor 200, an EKG or EEG monitor 204 and other types of equipment 208, such as the afore mentioned blood pressure sleeve 150, FIG. 9, are tagged using the above sensor assemblies 212, each sensor assembly preferably including a programmable memory that stores product information pertaining to the device that the sensor assembly is attached to. In essence, each of the sensor assemblies 212 are therefore used as tags wherein use of the interrogation device 30 and emission of the appropriate trigger signals identifies all equipment in a subject's room as displayed by the device. This method is useful in determining not only in locating or determining any equipment that is in the room, but also in expediting examination and physician rounds due to the potential time savings in not having to locate and otherwise retrieve equipment by means of the convenient tagging capability of the sensor assemblies 212.

The above method provides a means of traceability, for example, to determine whether or not a specific piece of equipment has been used in conjunction with a patient, settling, for example, potential billing and liability issues. Moreover, the physiological data obtained could be married with the equipment used. In a preferred version, the equipment (e.g. a vital signs monitor such as those manufactured under the tradename of Propaq® by Welch Allyn, Inc.) can wirelessly transmit physiological data directly to the sensor assembly for storage in the memory of the ASIC. This data can then subsequently be uploaded to the interrogation device when the trigger is transmitted to the sensor assembly.

Turning to FIG. 11, the sensor assembly can be used not only for external subject diagnosis, but also for determining a number of other patient-related conditions when used in connection with an endoscope or similar device (the insertion tube 220 being shown in the Fig.) that can be implanted within a subject's body cavity 224 (shown only schematically in FIG. 11), wherein a sensor assembly 228 can be attached to the exterior of the tube 220. Alternately, the insertion tube 220 can be used to actually implant a sensor assembly within the body cavity for a particular treatment and the assembly can then be subsequently removed at the end of the treatment.

Parts List For FIGS. 1-11

10 apparatus
20 sensor assembly
24 temperature responsive element
28 low power A/D circuit
30 interrogation device
32 emitter and receive circuitry
34 subject
35 unit serial number
36 antenna
38 emitter and receive circuitry
40 temperature computation algorithm(s)
42 serial interface
46 antenna
48 user interface
50 sensor assembly
54 first disposable supporting portion
58 second reusable portion
62 temperature responsive element
66 flexible strip
68 top surface
70 adhesive backing
74 leads
78 connection coupling leads
84 body portion
88 ASIC
92 wireless transmitter/receiver
96 antenna
100 sensor assembly
104 substrate
108 ASIC
112 thermistor
116 wireless transmitter
120 receiver
124 antenna
128 LCD
132 input controls
138 housing body
144 disposable sensor assemblies
148 arm
150 blood pressure sleeve
154 hook and loop fasteners
158 artery markers
162 facing side
168 gage housing
172 hose
176 coupling
180 sensor assembly
184 arm
200 vital signs monitor
204 EKG or EEG monitor
208 other equipment
212 sensor assemblies
220 insertion tube
224 body cavity
228 sensor assembly

While the present invention has been particularly shown and described with reference to the preferred mode as illustrated in the drawings, it will be understood by one skilled in the art that various changes in detailed may be effected therein without departing from the spirit and scope of the invention as defined by the following claims.

1. A wireless thermometer apparatus for measuring the body temperature of a subject, said apparatus comprising:
a sensor assembly including at least one temperature responsive element, said element producing a first signal upon detection of a change in temperature, a converter for converting the first signal into an electrical signal, and a transmitter for wirelessly transmitting the converted electrical signal upon demand; and
an interrogation device having a transmitter which wirelessly transmits a trigger signal wherein said sensor assembly does not transmit readings of said temperature responsive element until the interrogation device transmits the trigger signal, and in which said sensor assembly is disposed on a patient to enable temperature readings to be taken without significant delay, wherein at least a portion of said sensor assembly is disposable.

2. Apparatus as recited in claim 1, wherein said sensor assembly is insertable into a body cavity of said subject.

3. Apparatus as recited in claim 2, wherein said body cavity is the sublingual pocket.

4. Apparatus as recited in claim 2, wherein said body cavity is the axilla.

5. Apparatus as recited in claim 2, wherein said body cavity is the rectum.

6. Apparatus as recited in claim 2, wherein said body cavity is the earcanal.

7. Apparatus as recited in claim 1, wherein said sensor assembly is attachable to means which is inserted into the body of a subject.

8. Apparatus as recited in claim 7, wherein said body insertion means includes at least one of an endotracheal tube and an insertion tube.

9. Apparatus as recited in claim 1, wherein said sensor assembly is attachable to an EKG/EEG measuring apparatus.

10. Apparatus as recited in claim 1, including a wrappable portion which can be wrapped about a limb of a subject.

11. Apparatus as recited in claim 10, wherein said wrappable portion is disposable.

12. Apparatus as recited in claim 1, wherein said interrogation device includes control means for transmitting said signal to said sensor assembly at predetermined time intervals.

13. Apparatus as recited in claim 1, wherein said sensor assembly includes a programmable ASIC.

14. Apparatus as recited in claim 13, wherein said ASIC is reusable.

15. Apparatus recited in claim 1, wherein said sensor assembly is used with blood pressure measuring apparatus.

16. Apparatus as recited in claim 1, wherein said sensor assembly includes means for transmitting identification information along with temperature signals.

17. Apparatus as recited in claim 13, wherein said ASIC includes a programmable memory.

18. Apparatus as recited in claim 16, wherein at least one of device and subject related information can be stored into the programmable memory of said ASIC.

19. Apparatus as recited in claim 1, wherein said sensor assembly includes an antenna for receiving the transmit signal from the interrogation device.

20. Apparatus as recited in claim 19, wherein said antenna is made from silk-screen technology.

21. Apparatus as recited in claim 1, including encryption means for securing the data transmitted by said sensor assembly.

22. Apparatus as recited in claim 1, including at least two sensor assemblies for determining thermal gradients of a subject.

23. Apparatus as recited in claim 1, wherein said sensor assembly is flexibly attachable so as to assume the geometry of an object to which it is attached.

24. Apparatus as recited in claim 23, wherein at least a portion of said sensor assembly is manufactured from silk-screen technology.

25. Apparatus as recited in claim 1, wherein said sensor assembly is attachable to the skin of a subject.

26. Apparatus as recited in claim 23, wherein said sensor assembly is attachable to the skin of a subject.

27. Apparatus for measuring at least one physiological parameter of a subject, said apparatus comprising:
a sensor assembly including at least one physiological parameter responsive element, said responsive element producing a first signal upon detection of a change in physiological parameter, a converter for converting the first signal into an electrical signal, and a transmitter for wirelessly transmitting the converted electrical signal upon demand; and
an interrogation device having a transmitter which wirelessly transmits a trigger signal wherein said sensor assembly does not transmit readings of said physiological parameter responsive element until the interrogation device transmits the trigger signal, and in which said sensor assembly is disposed on a subject to enable temperature readings to be taken without significant delay, wherein at least a portion of said sensor assembly is disposable.

28. Apparatus as recited in claim 27, wherein at least one physiological parameter being measured is body temperature.

29. Apparatus as recited in claim 27, wherein said sensor assembly is insertable into a body cavity of said subject.

30. Apparatus as recited in claim 29, wherein said sensor assembly is attachable to means which is inserted into the body of a subject.

31. Apparatus as recited in claim 27, wherein said sensor assembly is attachable to at least one piece of equipment found in a subject's room.

32. Apparatus as recited in claim 27, wherein said interrogation device includes control means for transmitting said signal to said sensor assembly at predetermined time intervals.

33. Apparatus as recited in claim 27, wherein said sensor assembly includes a programmable ASIC.

34. Apparatus as recited in claim 33, wherein said ASIC is reusable.

35. Apparatus as recited in claim 27, wherein said sensor assembly includes means for transmitting device and subject identification information along with physiological parameter signals.

36. Apparatus as recited in claim 35, wherein at least one of device and subject related information can be stored into the programmable ASIC.

37. Apparatus as recited in claim 27, wherein said sensor assembly includes an antenna for receiving the transmit signal from the interrogation device.

38. Apparatus as recited in claim 37, wherein said antenna is made from silk-screen technology.

39. Apparatus as recited in claim 27, including encryption means for securing the data transmitted by said sensor assembly.

40. Apparatus as recited in claim 27, wherein said sensor assembly is flexibly attachable so as to assume the geometry of an object to which it is attached.

41. Apparatus as recited in claim 40, wherein said sensor assembly is attachable to the skin of a subject.

42. A method for identifying the location of medical equipment in a patient room, said method including the steps of:
attaching a sensor assembly to at least one piece of medical equipment, said sensor assembly including a programmable ASIC that includes information concerning said product stored therein, a wireless transmitter, a wireless receiver and an antenna for permitting bidirectional wireless communication;
selectively transmitting a trigger signal from an interrogation device in said patient room; and
in which said at least one sensor assembly transmits product information to said interrogation device only in response to said trigger signal.

43. A method for measuring a physiological parameter of a subject, said method comprising the steps of:
attaching a disposable sensor assembly to the body of a subject, said disposable sensor assembly including at least one physiological parameter sensor and circuitry responsive to a trigger signal;
selectively transmitting a trigger signal in the vicinity of said sensor assembly;
and in which said sensor assembly transmits a reading from said sensor only in response to reception of said trigger signal.
